(19) United States
(12) Patent Application Publication
(10) Pub. No.: US 2003/0092673 A1
(43) Pub. Date: May 15, 2003

(54) Substance and method for reduction of lipids and cholesterol

(76) Inventors: Henryk Struszczyk, Zgierz (PL); Elina Saynatjoki, Kuhmoinen (FI); Olli Kivekas, Tampere (FI); Harri Hakli, Vantaa (FI)

Correspondence Address: Connoly Bove Lodge & Hutz LLP, Suite 800, 1990 M Street, N.W., Washington, DC, 20036-3425, US

(21) Appl. No.: 09/921,657

(22) Filed: Aug. 6, 2001

Related U.S. Application Data
(63) Continuation of application No. 09/355,735, filed on Oct. 18, 1999, now abandoned.

(30) Foreign Application Priority Data
Feb. 6, 1997 (FI) 970499

(51) Int. Cl. A61K 31/722; C08B 37/08

(52) U.S. Cl. 514/55; 536/20

(57) ABSTRACT

A substance for reduction of absorption of lipids and for reduction of serum cholesterol content in mammals comprising microcrystalline chitosan. The microcrystalline chitosan can be administered orally in food products or in dose units containing a predetermined amount of microcrystalline chitosan. The microcrystalline chitosan has an average particle size not higher than 100 μm.

The invention relates to a substance and method for reduction of lipids by reducing their absorption in a living body. The invention also includes a substance and method for reduction of serum cholesterol content.

Lipids play an important role in many biological processes, and quite often such components as triglycerides, fatty and bile acids as well as cholesterol and other sterols act negatively for certain mammals, especially humans. The negative effects of lipids in many biological processes are described inter alia in: Laboratory Investigation; vol 39, pages 574-583, 1978, Biochimica Biophysica Acta, vol, 515, pages 163205, 1978 and International Journal of Pharmacy, vol. 74, pages 137-146, 1991. The subsequent digestion and absorption of lipids affects negatively the weight control as well as causes health problems.

Cholesterol, classified in lipid classification among precursor and derived lipids, an essential component of cell membranes and a precursor for steroid hormone synthesis and triglycerides, an important energy source, are transported as lipoproteins in the blood, Hyperlipoproteinemias are disturbances of lipid transport that result from accelerated synthesis or retarded degradation of lipoproteins. This abnormal phenomenon known as hyperlipidemia is detected by finding an elevated concentration of cholesterol and triglycerides in serum. High contents of serum lipoproteins are clinically very important, because they may cause two life-threatening problems: atherosclerosis and pancreatitis. Suitable reduction of the cholesterol-carrying lipoproteins, through diet and drugs, decreases a risk of myocardial infarction in subjects of hyperlipoproteinemia and hyperlipidemia.

The simplest way to prevent a high content of lipids in blood, including cholesterol, is the dietetic treatment in combination with special drugs. When a dietary therapy has failed to reduce the serum lipid concentration as well as in the case of hereditary hyperlipidemia and hyperlipoproteinemia, the use of lipid-lowering substances or special therapy is necessary. The serum exchange technique to lower the blood lipid concentration is well known from the monograph “Topic in Plasmophoresis”, published by ISAO Press., Cleveland, 1985. Another selected adsorption method by the lipid removal in blood perfusion is described in the paper Proc. Natl. Awd. Sci. Cuss., Vol. 8(110), p. 611. 1987.

Above techniques for reduction of the lipid concentration in blood are expensive and difficult to perform. Many absorbants, such as antibody ligands, anionic polymer ligands and nonion polymer ligands, are de scribed inter alia in the papers published in Journal of Polymer Science, Vol. 8, p. 263, 1952; Analytical Abstracts, Vol. 9, p.977, 1962; and Japanese Patent JP 62 244 442 (1987). The efficacy and susceptibility for practical applications of the above absorbants were very poor.

Application of cholestyramine being a synthetic cationic polymer under the trade name Dowex 1 having cationing tertiary amino groups for clinical use as a hypocholesterolemic drug is well known inter alia from the monograph “Chitin Derivatives in Life Science”, edited by S. Tokura and i. Acuma, 1992, by Japanese Society for Chitin/Chitosan, Sapporo, Japan. However, use of cholestyramine that is actually used to sequester phospholipids, fatty acids and cholesterol, has been questioned due to its toxicity because of possible link to colonic cancer in man and rats. These are well known from papers published in Proc. Soc. Biol. Med., Vol. 189(1), p. 13, 1988, and Amer. J. Clin. Nutr., Vol. 38(2), p. 278, 1983.

Initial chitosan (1,4-linked-β-D-glucosamine)—a standard material obtained through deacetylation of chitin—has a lipid-lowering action similar to those of cholestyramine but without any toxic effects. This antihypercholesterolemic and antihyperlipidemic action is a result of the inhibition of fat digestion by initial chitosan. The hypocholesterolemic activity of initial chitosan by oral administration has been found in rats and it is described in Nutr. Rep. Int., Vol. 18, p. 531, 1978.; Vol. 19, p. 327, 1979; Vol 20, p. 677, 1979; and Am. J. Clin. Nutr., Vol. 33, p. 787, 1980. Hypocholesterolemic quaternary ammonium salts of initial chitosan are also described in WO 92/06136.

Initial chitosan binds the fatty acids to form the corresponding complex salts by natural ionic bonds. After ingestion, the resulting salts bind additional lipids, due to hydrophobic interaction of triglycerides, fatty and bile acids, cholesterol and other sterols. Hydrochloric acid in the stomach does not hydrolyse chitosan-fatty acid salts, because this material hardly wets. The material grows in size during its transportation through the gastrointestinal tract and binds additional amounts of lipids. Bound dietary triglycerides escape the hydrolysis by lipase. As a summary, the chitosan, when ingested, promotes the binding and excretion of fatty materials including cholesterol, sterols and triglycerides. This process is described in U.S. Pat. No. 4,223,023 and in J. Nutr. Sci. Vitaminol., Vol. 38, p. 609, 1992. The studies of dietary action of initial chitosan applied in the diet in the amount of 3 to 6 g per day showed that the serum total cholesterol level significantly decreased, These studies are described in Biosc. Biotechn. Biochem., Vol. 57(9), p. 1439, 1993.

The anticholesterol action of initial chitosan are also described in Proceedings of 2nd Asia Pacific Chitin Symposium, Bangkok, 1996; Chitin Enzymology, Vol, 2, p. 55-63, 1996; Advances in Chitin Science, Vol. 1, p. 44B, J. Andre PubI., 1996.

The well-known anticholesterolically acting initial chitosan is, however, characterized by several disadvantages, such as insufficient effectivity to reduce the cholesterol level, especially in a long-term treatment, too high dimension of particles of initial chitosan used, as a result of the nature of form of this polymer, non-direct tableting behaviour as well as the usefulness of the form of initial chitosan in general.

Microcrystalline chitosan as a most useful form of chitosan with high developed intrinsic surface, controlled biodegradability and bioactivity, and high sorption capacity being much higher than with initial chitosan, is well known from Finnish Patent 83426 and the corresponding International Application, publication No. WO 91/00298; Journal of Applied Polymer Science, Vol 33, p. 177, 1987; British Polymer Journal, Vol. 23, p. 261, 1990; Advances in Chitin Science, Vol. 1, p. 482, J. Andre Pub[., Lyon, 1996.

The object of this invention is to effectively reduce the lipids absorption in mammals, especially humans. The object is attained by using microcrystalline chitosan in a form of microcrystalline powder and/or gel-like dispersion, orally administered in an amount suitable for effective reduction of the absorption of lipids.

According to the present invention, the substance for the reduction of lipids absorption in mammals comprises microcrystalline chitosan. The microcrystalline chitosan can be in the form of a powder with an average particle dimension lower than 100 μm and/or a gel-like dispersion with an average particle dimension lower than 100 μm. The material Is especially characterized by average molecular weight higher than 10.000 daltons, preferably 50.000 to 700.000 daltons and deacetylation degree higher than 60%, preferably 70 to 95%,

According to a preferred embodiment of the invention, the microcrystalline chitosan powder as a component in the substance for lipids reduction, formed preferably by air-spray drying, is characterized by water retention value of WRV not lower than 150%, preferably 200 to 300%, with high direct tabletting ability. According to a preferred embodiment of the invention, the microcrystalline chitosan powder as a component in the substance for lipids reduction, formed by lyophilization, is characterized by water retention value not lower than 200%, preferably 250 to 400%.

According to a preferred embodiment of the invention, the microcrystalline chitosan gel-like dispersion as a component in the substance for lipids reduction, containing more than 0.1 wt-% of microcrystalline polymer, preferably 1 to 4 wt-%, is characterized by water retention value not lower than 500%, preferably 800 to 5000%.

The object of this invention is also to effectively reduce the serum cholesterol level in mammals, especially humans. The object is attained by using microcrystalline chitosan with specified properties in the form of a gel-like dispersion and/or powder, orally administered in an amount suitable to give an effective reduction of the serum cholesterol content.

According to the present invention, the substance for the reduction of serum cholesterol content in mammals by chitosan comprises microcrystalline chitosan, The microcrystalline chitosan can be in the form of a powder with an average particle dimension lower than 80 μm and/or a gel-like dispersion with an average particle dimension lower than 200 μm. The material is especially distinguished by average viscometric molecular weight higher than 1000 daltons, preferably 10,000 to 300,000 daltons, and a deacetylation degree in the range of 60 to 99%, preferably 80 to 95%.

According to a preferred embodiment of the invention, the microcrystalline chitosan powder as a component in the substance for serum cholesterol level reduction, produced preferably by air-spray drying and/or lyophilization, is distinguished by a water retention value not lower than 100%, preferably 150 to 250%

According to a preferred embodiment of the invention, the microcrystalline chitosan gellike dispersion as a component in the substance for serum cholesterol level reduction, containing more than 0.5 wt-% of microcrystalline polymer, preferably 1 to 3 wt-%, is distinguished by a water retention value not lower than 300%, preferably 500 to 2000%.

According to a preferred embodiment of the invention, the microcrystalline chitosan is administered orally in the form of a gel-like dispersion and/or powder and/or tablets and/or capsules, together with and/or independently of food or as the additives of food.

The benefit concerned with the use of microcrystalline chitosan as the lipid and cholesterol binder is concerned with its ability to exist in a form of powder or/and gel-like dispersion, characterized by a very small average particle size which is not higher than 100 μm, preferably not higher than 80 μm and usually 10 to 65 μm, which sizes are impossible to produce by mechanical grinding of initial chitosan. The above size of particles has effect directly on the lipid and cholesterol binding capacity.

Finally, the microcrystalline chitosan, especially in the form of air-spray dried powder, is the only chitosan form distinguished by direct tabletting behaviour connected with the ability of the individual particles of this polymer to form hydrogen bonds.

The invention will be described in the following more closely with reference to the accompanying drawings, where

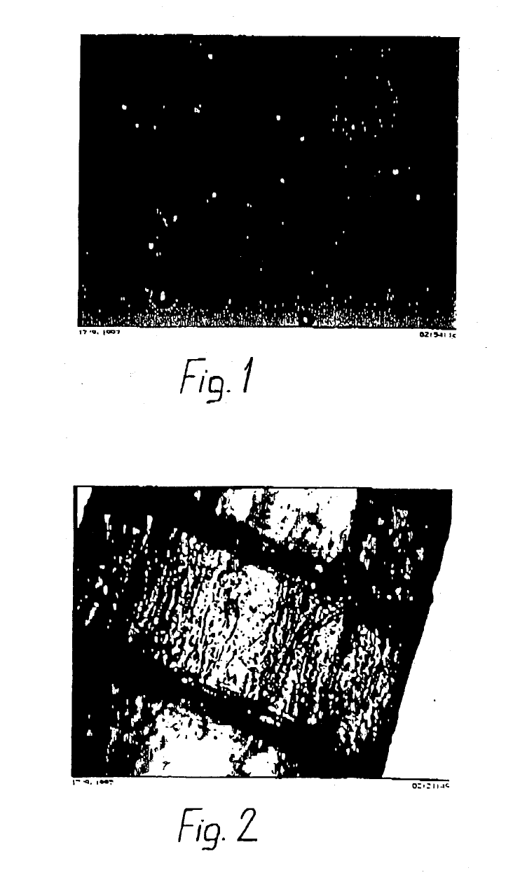

FIG. 1 is a microscopic photograph of microcrystalline chitosan powder, magnification 62 times, and

FIG. 2 is a microscopic photograph of initial chitosan, magnification 40 times, used as well known agent for reduction of cholesterol content.

Microcrystalline chitosan, the agent responsible for the advantageous effects is prepared by aggregating the glucosamine macromolecules from a solution of chitosan in an acid by introducing an alkaline solution and stirring. The method is known as such, and reference is made to the documents mentioned above.

The use of microcrystalline chitosan characterized by high developed intrinsic surface as a serum cholesterol content agent applied to mammals according to the invention is concerned with its very high susceptibility to complex the cholesterol, especially low density cholesterol, as well as triglycerides. The microcrystalline chitosan according to the invention additionally binds the lipids, such as fatty acids and bile acids The behaviour of cholesterol and triglyceride complexing by microcrystalline chitosan (FIG. 1) with properties according to the invention is extraordinarily higher than well-known initial chitosan (FIG. 2) used previously. There are extraordinary properties of microcrystalline chitosan, such as high developed intrinsic surface (FIG. 1), high susceptibility for complexation of cholesterol and triglycerides, biocompatibility, high ability for ionic bonds creation and high ability for hydrogen bonds formation that are superior to well-known initial chitosan.

The microcrystalline chitosan according to the invention is characterized at first by its susceptibility to form suitable complexes with cholesterol, other sterols and triglycerides due to its high developed intrinsic surface, covered also by bound fatty acids producing suitable salts with this microcrystalline chitosan by natural ionic bonds. A higher amount of bound fatty acid salts supports also much more the complexation of cholesterol, other sterols and triglycerides, in comparison with known initial chitosan. Hydrochloric acid in the stomach, not hydrolysing microcrystalline chitosan complexes with cholesterol and triglycerides as well as salts with bound fatty acid, disperses, swells and emulgates these complexes. Thereafter, they grow in size during their transportation through the gastrointestinal tract. This complexed microcrystalline chitosan, due to its structural and surface advantages, also binds additional amounts of cholesterol, triglycerides and lipids, The complexation of cholesterol, other sterols and triglycerides by microcrystalline chitosan according to the invention is much higher and faster with beneficial effectivity, in comparison with well-known initial chitosan. The complexed cholesterol, other sterols and triglycerides with accompanying lipids joined with microcrystalline chitosan are excreted from the organism.

Microcrystalline chitosan according to the invention is characterized by selective action against two different forms of cholesterol. This microcrystalline chitosan complexes with the low density cholesterol of LDL with a high effectivity, producing a negative effect on hypercholesterolemic and hyperlipidemic phenomena, and often slightly increasing the high density cholesterol of HDL, having a positive action for a living organism, such as a human.

Microcrystalline chitosan used according to the method of invention is characterized by a special range of average molecular weight with highest effectivity for complexation of cholesterol, other sterols and triglycerides. The average molecular weight of microcrystalline chitosan useful for reduction of serum cholesterol content is much lower than for binding of lipid and it is not lower than 1000 daltons, especially in the range of 10,000 to 300,000 daltons with deacetylation degree in the range of 60 to 99%, especially 80 to 95%. The most important behaviour of microcrystalline chitosan according to the invention is continuous reduction of serum cholesterol content and stability of this process, in contrast to the initial chitosan where this phenomenon is not stable and usually is characterized by increase during its application.

Microcrystalline chitosan in the form of an air-spray dried powder with an average molecular weight of Mv=50.000 to 300.000 daltons and deacetylation degree of 70 to 90% has clinically reduced the total serum cholesterol content during 5 months of its oral administration by 16 to 25%, already in comparison to an initial content measured after 3 months from starting the treatment, depending on the individual test person behaviour. After the same time the serum triglyceride content was reduced during two months by 21 to 43% with the same test persons. The known initial chitosan at similar conditions has reduced the total serum cholesterol by 10% and triglycerides by 11 to 14% only.

The advantage of microcrystalline chitosan use according to the invention is connected with variety of its possible forms from gel-like dispersion to powder. No special diet is necessary to be used together with microcrystalline chitosan.

The following methods of the determination of microcrystalline chitosan properties were used:

average molecular weight of microcrystalline chitosan: according to a method described in “Chitin”, Pergamon Press, New York, 1977,

deacetylation degree of microcrystalline chitosan: according to a method described in the International Journal of Biological Macromolecules, Vol. 2, p. 115, 1980,

water retention value of microcrystalline chitosan: according to a method described in the Cellulose Chemistry and Technology, Vol. 11, p. 633, 1977,

fatty acids binding capacity for chitosan and microcrystalline chitosan: according to the modified method described in the Carbo. hydrate Polymers, v.22, p.117, 1993. The binding capacity in percentage was calculated as the ratio of amount of fatty acid salt in the form of sodium undecylenic bound by chitosan to the total amount of this salt used for testing.

average particle size of microcrystalline chitosan particles: according to the microscopic method with computer estimation.

The content of total serum cholesterol and HDL cholesterol as well as triglyceride in humans and rabbits were determined according to the standards

The invention will be explained further in the following examples which do not restrict the scope of the appended claims.

### EXAMPLE 1

Microcrystalline chitosan gel-like dispersion characterized by polymer content of 4.22 wt-%, average molecular weight of Mv=165.000 and deacetylation degree of 82% and water retention value of WRV 768% was subjected to the air-spray drying using aqueous dispersion containing 1.8 wt-% of polymer, incoming air temperature of 185° C., out coming air temperature of 80° C. and drying rate of 0.04 l per minute of dispersion.

The microcrystalline chitosan powder obtained was characterized by polymer content of 91.6 wt-%, WRV=215%, Mv=123.000 daltons and average particle size of 48 μm. This microcrystalline chitosan was distinguished by binding capacity for sodium undecylenic after 15 min of treatment of 66%. The capsules containing 0.383 g obtained microcrystalline chitosan powder were used for clinical tests in vivo A daily dose for the group of overweight persons with a weight ranged from 80 to 120 kg was 8 capsules with a total amount of microcrystalline chitosan of 3.064 g. No special diet was used for the testing persons. During 2 weeks of test, the persons were slimmed approximately 2 kg down.

### EXAMPLE 2

Microcrystalline chitosan gel-like dispersion with properties described in Example 1 was used for binding the fatty acids. The binding capacity of this dispersion after 15 min of treatment was 78%.

### EXAMPLE 3

Microcrystalline chitosan gel-like dispersion characterized by polymer content of 3.08 wt-%, average molecular weight of Mv=277.000 daltons and deacetylation degree of 83% with water retention value of WRV=1096% for gel was subjected to the air-spray drying with incoming air temperature of 210° C., outcoming air temperature of 70° C. with a drying rate of 72 ml dispersion per minute. The air-spray dried powder obtained was characterized by polymer content of 92.6 wt-%, deacetylation degree of 83%, water retention value of WRV=288% and average molecular weight of Mv=185.000 daltons as well as average particle size of 40 μm.

This microcrystalline chitosan powder was characterized by binding capacity of 71.8% of sodium undecylenic after 15 min of treatment.

### EXAMPLE 4

Microcrystalline chitosan gel-like dispersion with properties described in Example 3 was used for binding the fatty acids

The binding capacity for sodium undecylenic was 766% after 15 min of treatment.

### EXAMPLE 5

Microcrystalline chitosan gel-like dispersion characterized by polymer content of 4.72 wt-%, average molecular weight of Mv=387.000 daltons, deacetylation degree of 81%, WRV=809% was subjected to the air-spray drying with incoming air temperature of 180° C., outcoming air temperature of 70° C., drying rate of 60 ml dispersion per minute.

The air-spray dried powder obtained was characterized by polymer content of 94.5 wt-%, WRV=273% and average molecular weight of 101.000 daltons with average particle size of 35 μm. This microcrystalline chitosan was characterized by binding capacity of 64.6% after 15 minutes of treatment.

### EXAMPLE 6

Microcrystalline chitosan gel-like dispersion with properties described in Example 5 was used for binding the fatty acids.

The binding capacity for sodium undecylenic after 15 minutes of treatment was 86.6%.

### EXAMPLE 7

Microcrystalline chitosan in a form of gel-like dispersion characterized by polymer content of 4.68 wt-%, WRV=794%, deacetylation degree of 87%, Mv=520.000 daltons was subjected to the air-spray drying as in Example 1.

The air-spray dried powder obtained was characterized by polymer content of 94.7 wt-%, WRV=300%, Mv=180.000 daltons and deacetylation degree of 87.4% with binding capacity after 15 min of treatment of 73.6%.

### EXAMPLE 8

Microcrystalline chitosan gel-like dispersion with properties as in Example 1 was used for binding of fatty acids. The binding capacity for sodium undecylenic after 15 min. of treatment was 86.2%.

### EXAMPLE 9

Microcrystalline chitosan in a form of gel-like dispersion characterized by polymer content of 4.94%, WRV=776%, deacetylation degree of 86.8% and Mv=503,000 daltons was subjected to the air-spray drying as in Example 3.

The microcrystalline chitosan powder obtained was characterized by polymer content of 83.1%, WRV=310%, deacetylatlon degree of 84% and Mv=422.000 daltons and binding capacity after 15 min of treatment of 82.6%.

### EXAMPLE 10

Microcrystalline chitosan gel-like dispersion with properties as in Example 9 was used for binding of fatty acids. The binding capacity for sodium undecylenic was 86.8%.

### EXAMPLE 11

Lyophilized microcrystalline chitosan with polymer content of 89.1 wt-%, WRV=132%, deacetylation degree of 71% and Mv=330.000 dattons was used for fatty acid bindings. Its binding capacity after 15 min of treatment for sodium undecylenic was 81%.

### EXAMPLE 12

1.0 weight part of microcrystalline chitosan gel-like dispersion with properties as in Example 3 was homogenized for 5 min with 50 rpm with 10 weight parts of yogurt to obtain the stable dispersion having specific behaviour of nonfatty dairy product causing the slimming process.

### EXAMPLE 13

1.5 weight parts of microcrystalline chitosan gel-like dispersion with properties as in Example 3 was homogenized with 10 weight parts of low caloric butter for 30 min with 30 rpm. The stable butter with specific behaviour of low fatty butter causing reduction of introduced fatty component to the human body was obtained.

### EXAMPLE 14

0.7 weight parts of microcrystalline chitosan gel-like dispersion with properties as in Example 10 was homogenized for 20 min with 150 rpm with 15 weight parts of white cheese. The stable modified white cheese with specific behaviour of low fatty dairy product causing reduction of Introduced fatty component to the human body was obtained.

### EXAMPLE 15

The rabbits were fed with a fat rich feed using a normal feeding frequency during 2 months of test. After the first month of test, the microcrystalline chitosan in the form of a gel-like dispersion characterized by water retention value of WRV=1030%, average viscometric molecular weight of Mv=2.46×10⁵ daltons, deacetylation degree of DD=74% average particle dimension of 60 μm and polymer content of 3.0 wt-%, was added to the rabbit food in an amount of 1 wt-% on dry weight. The test was carried out for the next month. The total content of serum cholesterol, triglycerides and HDL cholesterol was determined at the beginning of microcrystalline chitosan application and at the end of the test of 1 month after microcrystalline chitosan application. The results of determinations, in percentage to initial amount, are presented in the table below.

TABLE 1
Reduction of serum total and HDL cholesterol as well as
triglyceride content.

| | Percentage of change, in comparison to initial | | |
|---|---|---|---|
| | content, for: | | |
| Symbol of rabbit | total cholesterol | HDL cholesterol | triglycerides |
| A | −28.3 | −43.6 | −55.4 |
| B | −13.9 | −54.9 | −38.6 |
| C | −7.0 | +4.3 | −69.6 |

−= reduction, += increase

### EXAMPLE 16

The rabbits were fed as in Example 15 during 2 months of test, After the first month of test, the high molecular weight microcrystalline chitosan in the form of a gel-like dispersion, characterized by water retention value of WRV=1030%, Mv=2.46×10⁵ daltons, OD=74%, average particle dimension of 45 μm and polymer content of 2.1 wt-%, was added to the rabbit food in an amount of 1 wt-% on dry weight. The test was carried out for the next month. The serum determinations, in percentage to initial amount, are presented in the table below.

TABLE 2
Reduction of serum total and HDL cholesterol as well as
triglyceride content.

| | Percentage of change, in comparison to initial | | |
|---|---|---|---|
| | content, for: | | |
| Symbol of rabbit | total cholesterol | HDL cholesterol | triglycerides |
| D | −51.3 | −25.7 | −73.0 |
| E | −31.7 | −16.9 | −67.9 |
| F | −28.4 | +15.4 | −82.7 |

### EXAMPLE 17

The rabbits were fed as in Example 1 during 2 months of test. After the first month of test, the low molecular weight microcrystalline chitosan in the form of a gel-like dispersion, characterized by WRV=1100%, Mv=1.28×10⁵ daltons, DD=73%, average particle dimension of 40 gm and polymer content of 1.9 wtv %, was added to the rabbit food in an amount of 1 wt-% on dry weight. The test was carried out for the next month. The serum determinations, in percentage to initial amount, are presented in the table below.

TABLE 3
Reduction of serum total and HDL cholesterol as well as
triglyceride content.

| | Percentage of change, in comparison to initial | | |
|---|---|---|---|
| | content, for: | | |
| Symbol of rabbit | total cholesterol | HDL cholesterol | triglycerides |
| G | −53.7 | −43.5 | −19.4 |
| H | −37.3 | −48.0 | −47.4 |

### EXAMPLE 18

Three test persons with overweight were clinically tested for reduction of serum total cholesterol content. Two of them were also tested for reduction of serum triglycerides. They used a normal diet for 8 months. Microcrystalline chitosan in the form of an air-spray dried powder, characterized by WRV=200%, Mv=1.27×10⁵ daltons, DD=87% average particle dimension of 25 μm and moisture content of 7%, administered orally as capsules containing 400 mg in a dose of 3.2 mg per day of microcrystalline chitosan powder was used for test. The values of serum total cholesterol and triglyceride content during the testing time is presented in the Tables 4 and 5 below.

TABLE 4
Reduction of human serum total cholesterol content.

| | Total cholesterol content, mmol/l, | | | | | |
|---|---|---|---|---|---|---|
| | as function of time in months | | | | | |
| Symbol of person | 0 | 3 | 4 | 5 | 6 | 8 |
| A | 11.5 | — | 6.4 | — | 4.9 | 4.7 |
| B | 8.8 | 6.3 | — | 5.4 | — | 5.3 |
| C | 7.9 | 5.6 | — | 4.7 | — | 4.2 |

0 months: time of starting the administration of microcrystalline chitosan

TABLE 5
Reduction of human serum triglyceride content.

| Symbol of | Triglyceride content, mmol/l, as a function of time in months | | |
|---|---|---|---|
| test person | 0 | 3 | 5 |
| B | 4.7 | 2.8 | 1.6 |
| C | 2.5 | 1.9 | 1.5 |

0 months: time of starting the administration of microcrystalline chitosan

As mentioned above, the substance causing the reduction of the absorption of lipids and/or serum cholesterol content can be formed into a suitable composition, which can be administered separately as a predetermined dose, e.g. the effective amount of microcrystalline chitosan can be combined with inert pharmaceutically acceptable carriers. The substance can also be incorporated into food, especially into food products containing lipids and/or cholesterol, whereby the microcrystalline chitosan will be administered together with the food. The microcrystalline chitosan can be incorporated in the composition as such or in the form of a suitable complex.

Further, apart from human body, the substance can be administered also to the body of a non-human mammal, such as domestic animals and pets, if there is a necessity to reduce the absorption of lipids and/or serum cholesterol content in these animals. All above-mentioned ways and compositions of oral administration can be used also for this alternative.

1. Substance for reduction of absorption of lipids in mammals, the substance comprising chitosan, characterized in that the chitosan is microcrystalline chitosan.

2. Substance as claimed in claim 1, characterized in that the microcrystalline chitosan has an average particle size not higher than 100 μm.

3. Substance as claimed in claim 1 or 2, characterized in that the microcrystalline chitosan is in the form of a powder with an average particle dimension lower than 100 μm and/or a gel-like dispersion with an average particle dimension lower than 100 μm.

4. Substance as claimed in claim 1, 2 or 3, characterized in that the microcrystalline chitosan is characterized by average molecular weight higher than 10.000 daltons, preferably 50.000 to 700.000 daltons and deacetylation degree higher than 60%, preferably 70 to 95%

5. Substance as claimed in any of the preceding claims, characterized in that the microcrystalline chitosan powder, formed preferably by air-spray drying, is characterized by water retention value of WRV not lower than 150%, preferably 200 to 300%, with high direct labletting ability.

6. Substance as claimed in any of the claims 1 to 4, characterized in that the microcrystalline chitosan powder, formed by lyophilization, is characterized by water retention value not lower than 200%, preferably 250 to 400%.

7. Substance as claimed in any of the claims 1 to 4, characterized in that the microcrystalline chitosan gel-like dispersion containing more than 0.1 wt-% of microcrystalline polymer, preferably 1 to 4 wt-%, is characterized by water retention value not lower than 500%, preferably 800 to 5000%.

8. Substance for reduction of serum cholesterol content in mammals, the substance comprising chitosan, characterized in that the chitosan is microcrystalline chitosan.

9. Substance as claimed in claim 8, characterized in that the microcrystalline chitosan has an average particle size not higher than 100 μm.

10. Substance as claimed in claim 8, characterized in that the microcrystalline chitosan is in the form of a powder with an average particle size smaller than 80 μm and/or a gel-like dispersion with an average particle size smaller than 200 μm.

11. Substance as claimed in claim 8, 9 or 10, characterized in that the microcrystalline chitosan is distinguished by an average viscometric molecular weight higher than 1000 daltons, preferably in the range of 10,000 to 300,000 daltons, and a deacetylation degree in the range of 60 to 99%, preferably 80 to 95%.

12. Substance as claimed in any of the claims 8 to 11, characterized in that the microcrystalline chitosan powder produced preferably by air-spray drying and/or lyophilization is distinguished by a water retention value not lower than 100%, preferably 150 to 250%.

13. Substance as claimed in any of the claims 8 to 11, characterized in that the microcrystalline chitosan gel-like dispersion containing more than 0.5 wt-% of microcrystalline polymer, preferably 1 to 3 wt-%, is distinguished by a water retention value not lower than 300%, preferably 500 to 2000%.

14. Substance as claimed in any of the preceding claims, characterized in that it is a food product containing microcrystalline chitosan as an additive.

15. Substance as claimed In any of the preceding claims, characterized in that it is in the form of a dose to be administered orally, such as in the form of a tablet or capsule, possibly combined with pharmaceutically acceptable carriers.

16. Method for reduction of absorption of lipids in mammals, especially humans, the method comprising administering orally chitosan, characterized in that microcrystaliine chitosan as claimed in any of the claims 1 to 7 is administered orally in a suitable composition.

17. Method as claimed in claim 16, characterized in that the microcrystalline chitosan is administered in dose units of predetermined content of the microcrystalline chitosan in the form of a gel-like dispersion and/or powder.

18. Method for reduction of serum cholesterol content in mammals, especially humans, the method comprising administering orally chitosan, characterized in that the microcrystalline chitosan as claimed in any of the claims 8 to 13 is administered orally in a suitable composition.

19. Method as claimed in claim 18, characterized in that the microcrystalline chitosan is administered in dose units of predetermined content of the microcrystalline chitosan in the form of a gel-like dispersion and/or powder.
